(19) United States
(12) Patent Application Publication
(10) Pub. No.: US 2004/0003455 A1
(43) Pub. Date: Jan. 8, 2004

(54) System for protection from falls

(76) Inventor: Maximilian E. Davidson, Herzlia (IL)

Correspondence Address: Eitan Pear, Latzer & Cohen Zedek, Suite 1001, 10 Rockefeller Plaza, New York, NY, 10020, US

(21) Appl. No.: 10/380,008

(22) PCT Filed: Aug. 30, 2001
(86) PCT No.: PCT/IL01/00821

(30) Foreign Application Priority Data
Sep. 11, 2000 (IL) 138398

(51) Int. Cl. A41D 1/00

(52) U.S. Cl. 2/455

(57) ABSTRACT

A wearable inflatable system (20) adapted to protect a falling body, the system (20) includes at least one inflatable element (22, 24, 26) wearable on a body, which inflates and adapts to adjust a trajectory of the body during a fall, and/or to adjust the alignment of a back and a hip of a body during a fall.

### FIELD OF THE INVENTION

The present invention relates to a device which protects against injury in general and, specifically, against injuries which result from falls.

### BACKGROUND OF THE INVENTION

Falling or stumbling many times results in injuries such as broken bones. Elderly people, who may suffer from osteoporosis or other bone weakening diseases, are especially prone to such breaks and bruises. In order to prevent these injuries, prior art devices provide airbags that may be worn as belts or garters. An exemplary prior art cushion device 10 is illustrated in FIG. 1. Although, these devices may provide cushioning protection, the protection is limited.

### SUMMARY OF THE INVENTION

An object of the present invention is to provide a system which may sense an upcoming fall and which may provide means to avoid injury in the event of such a fall. In some embodiments the invention may be applicable for both human and animals.

There is therefore provided, in an embodiment of the present invention, a wearable inflatable system adapted to protect a falling body, the system including at least one inflatable element wearable on a body, which inflates and adapts to adjust a trajectory of the body during a fall.

There is further provided, in an embodiment of the present invention, a wearable inflatable system adapted to protect a falling body, the system including at least one inflatable element wearable on a body, which inflates and adjusts the alignment of a back and a hip of a body during a fall.

The inflatable element may b worn in a hip area of the body, and may support the hip area. The inflatable element may additionally be adapted to geometrically adjust the trajectory. Moreover, the inflatable element may include at least two inflatable cells. The two cells maybe be interconnected or they may be separated.

The inflatable element may also include a first inflatable element adapted to be worn on a waist of the body and a second inflatable element connectable to the first element and adapted to wear on a back of said body. The second element may be adapted to inflate along the back, and may be adapted to generally align the back during a fall.

The inflatable element may further include a third inflatable element connectable to the first element and adapted to wear on a buttocks area of the body. This third element may be adapted to inflate in the buttocks area, and may be adapted to provide cushioning of the buttocks. Furthermore, the third inflatable element may be adapted to cause the body to assume a sitting position.

The system may further include a gas source for inflating the at least one element, and it may include a controller for sensing an indication of a fall and, in response to one or more signals indicative of the fall, cause inflation of the at least one inflatable element. The controller may include one or more sensors for sensing a fall. The sensors may be a speedometer, an accelerometer, a gyroscope, a physiological meter, a heart monitor, and/or a tilt monitor.

Additionally, the one or more signals may be in response to sensed data, where the data may be tilt, speed, distance, perspiration, and/or pulse rate. The controller may include a processor.

### BRIEF DESCRIPTION OF THE DRAWINGS

The present invention will be understood and appreciated more fully from the following detailed description taken in conjunction with the appended drawings in which:

FIG. 1 is a schematic illustration of a prior art cushion device; and

FIG. 2 is a schematic illustration of a fall adjustment device according to an embodiment of the present invention

### DETAILED DESCRIPTION OF THE PRESENT INVENTION

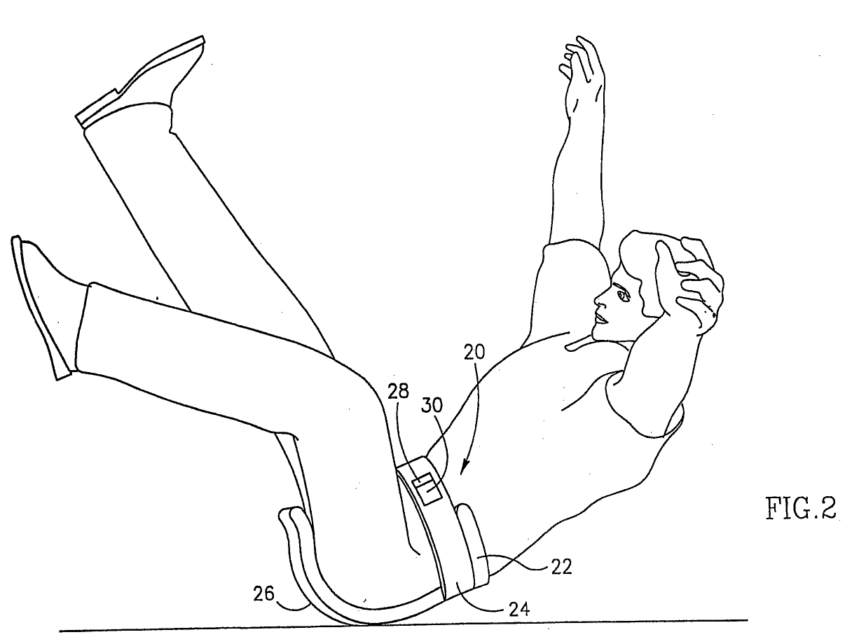

Reference is now made to FIG. 2, an illustration of a trajectory alignment system 20, constructed and operative according to an embodiment of the present invention. System 20 comprises rear inflatable element 22, waist inflatable element 24, lower inflatable element 26, controller 28 and one or more gas generators 30. In some embodiments, alignment system 20 is worn around the waist or hip area as one would wear a belt or girdle.

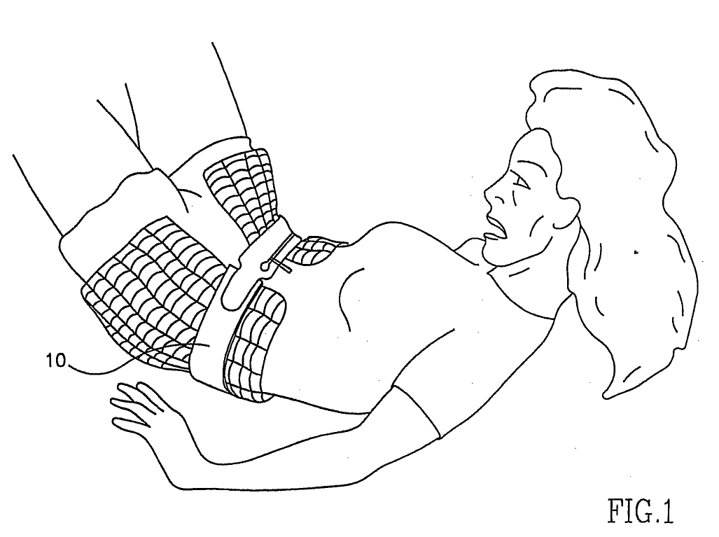

In contrast to prior art (FIG. 1) which only provides cushioning to various body parts with the aim of buffering impact, alignment system 20 may change the orientation of the falling body, thus creating a more proper falling position and avoiding possible injury. Just as Judo teaches a “proper” falling angle, likewise, via inflatable elements 22, 24 and 26, alignment system 20 may adjust the trajectory of the falling body, thereby avoiding dangerous falling positions.

Geometrically speaking, the human body, as well as any other body, is comprised of a number of segments (i.e. legs, arms, torso, etc.) interconnected by muscle. In the event of a fall, these segments behave in definite ways. For example, when a person's body is erect and in a forward motion (i.e. walking), losing contact at the bottom of the body (i.e. the heel slipping) will cause the body to fall flat on the back and/or side. However, when performing the same kind of motion (e.g. walking) but with the body bent forward, losing contact at the bottom of the body will cause the body to fall on the buttocks. Falling on the buttocks is kinder toward the body, and puts less stress on the hip, which is an especially fragile part of an elderly person's body.

According to an embodiment for operation of the present invention, when a fall is sensed, elements 22, 24 and 26 may be inflated. Element 24 may create a cushion around the hip area. Element 22 may cause the back of the falling body to straighten, thus aligning the trajectory of the fall. Element 26 inflates on the underside of the buttock area, and may cause the legs to curl under, causing the buttocks to jut out, further aligning the trajectory of the fall. Additionally, element 26 may provide protection to the buttocks area, further reducing the impact of the fall.

Thus, because the geometry of the fall may be modified, instead of falling flat on the back or the side, the body may receive a thrust of adjustment from elements 22, 24 and/or 26, along the hip area, the back, and/or the buttocks. This may result in a correction in the trajectory of fall, causing the body to fall on the buttocks area. Moreover, rearranging the trajectory of the fall may help other body parts, such as the shoulder joint and the knee joint, to avoid contact with the ground, and thus also may prevent possible injury.

Inflatable elements 22, 24 and 26 may be ergonomically designed to restrain the geometrical shape of the joint and/or body (e.g. hip joint) by applying pressure to those body parts. Furthermore, elements 22, 24 and 26 may provide physical support of those body parts and/or may modify the geometrical shape of the body, thus cushioning the fall and/or contact between the joint/body and any surface. Yet further, elements 22, 24 and 26 may be inflated relatively simultaneously or sequentially, with element 22 being inflated first, then 24, followed by 26. Sequential inflation may facilitate control over the modification of the body's trajectory.

Although the invention here is described as comprising separate inflatable elements, it is understood that the present invention may comprise an equivalent single inflatable element, or other configuration, which may provide the same functions and still fall within the scope of the present invention. An example of such may be a single inflatable element with interconnected compartments, or with two or more inflatable cells. The cells may be interconnected, or may be separate from each other.

By way of illustration, the present invention may comprise an element with internal inflatable sections and be geometrically adapted to provide the functions described herein. Each such section may be interconnected to the other sections or may be separated.

Controller 28 may sample movements of the body at predetermined intervals (e.g. every 0.3 msec). Controller 28 may additionally analyze movement by using a CPU and logic circuitry (not shown) and receive input(s) from one or more sensors (not shown). The sensors may comprise one or more of the following: a speedometer, an accelerometer, a gyroscope, a physiological meter (e.g. heart monitor), a tilt monitor, etc. The inputs may be tilt, speed, distance, perspiration, pulse rate, etc. A program inside the CPU may compare the inputs to data representative of normal movements. The resultant comparison may be used to detect an anomaly in movement.

Once an anomaly is detected, a signal may be sent to gas generator 30, causing elements 22, 24, and 26 to inflate. Gas generators 30 may be ‘cold type’. The cold type gas generator typically provides a relatively smaller shock, thus sparing the body impact from inflation. Additionally, cold type generators provide relatively quick gas discharge. The discharged gas may inflate the one and/or more inflatable elements (22, 24 and/or 26).

Below is detailed an example of operation according to an embodiment of the present invention: An elderly person slips on a wet surface and falls. The height of the hip is one (1) meter and the speed of the fall is 10 m/sec. The distance (1 meter) may be ‘traveled’ in 100 msec. (e.g. 100 msec until contact with the ground.)

If controller 28 is sampling movements every 0.3 msec, a fall may be detected in 6 msec: (sampling 10 times—3 msec.; analyzing—2 msec.; triggering—1 msec). Gas generator 30 releases the gas, causing inflation of elements 22, 24 and 26. The approximate time from release of gas to completion of inflation may be approximately 25-30 msec. Total time from detecting a fall until the fall is modified and protected: 36 msec.

Thus, since total time from the start of the fall until contact with the surface is 100 msec, alignment system 20 may be fully operable in sufficient time to prevent injury. It is noted that these figures are exemplary only and may differ for each event and according to the embodiment.

This invention may be especially useful for the elderly. In alternative embodiments, the present invention is adaptable for application to animals. Although the present invention discusses wearing system 20 in the hip areas, in alternative embodiments, the present invention may be applicable to other body joints. In such instances system 20 is adaptable for wearing on other parts of the body, with the elements adapted to fit the particular body part. The adapted elements may be triggered generally simultaneously as elements 22, 24 and 26.

The present invention may additionally comprise a belt or harness for attaching system 20 to the body.

Thus, the present invention may be adapted to:

modify the trajectory of the fall by changing the position of the body parts,

restrain the body part against changes in its internal geometry, and

protect during a fall.

It will be appreciated by persons skilled in the art that the present invention is not limited by what has been particularly shown and described herein above. Rather the scope of the invention is defined by the claims that follow:

What is claimed is:

1. A wearable inflatable system adapted to protect a falling body, the system comprising:
at least one inflatable element wearable on a body, which inflates and adapts to adjust a trajectory of said body during a fall.

2. A wearable inflatable system adapted to protect a falling body, the system comprising:
at least one inflatable element wearable on a body, which inflates and adjusts the alignment of a back and a hip of a body during a fall.

3. A system according to any of the previous claims, wherein said at least one element is worn in a hip area of said body.

4. A system according to claim 3,wherein said at least one element supports said hip area.

5. A system according to any of the previous claims, wherein said at least one element is adapted to geometrically adjust said trajectory.

6. A system according to any of the previous claims, wherein said at least one element comprises at least two inflatable cells.

7. A system according to claim 6, wherein said at least two inflatable cells are interconnected.

8. A system according to claim 6, wherein said at least two inflatable cells are separated.

9. A system according to any of the previous claims, wherein said at least one element comprises;
a first inflatable element adapted to be worn on a waist of said body; and
a second inflatable element connectable to said first element and adapted to wear on a back of said body,
said second element adapted to inflate along said back, and adapted to generally align said back during said fall.

10. A system according to claim 9, wherein said at least one element further comprises:
a third inflatable element connectable to said first element and adapted to wear on a buttocks area of said body, said third element adapted to inflate in said buttocks area, and adapted to provide cushioning of said buttocks.

11. A system according to claim 10, wherein said third inflatable element is adapted to cause said body to assume a sitting position.

12. A system according to any of the previous claims, further comprising a gas source for inflating said at least one element.

13. A system according to any of the previous claims, further comprising a controller for sensing an indication of fall and, in response to one or more signals indicative of said fall, for causing inflation of said at least one element.

14. A system according to claim 13, wherein said controller comprises one or more sensors for sensing said fall.

15. A system according to claim 14, wherein said sensors are selected from a group consisting of: a speedometer, an accelerometer, a gyroscope, a physiological meter, a heart monitor, and a tilt monitor.

16. A system according to claim 13, wherein said one or more signals are in response to a sensed data selected from a group consisting of: tilt, speed, distance, perspiration, and pulse rate.

17. A system according to claim 13, wherein said controller comprises a processor.
